(19) United States
(12) Patent Application Publication
(10) Pub. No.: US 2026/0294479 A1
(43) Pub. Date: Oct. 1, 2026

(54) SYSTEMS AND METHODS FOR PROVIDING BONE MOVEMENT

(71) Applicant: DePuy Synthes Products, Inc., Raynham, MA (US)

(72) Inventor: Jacob Hunter LOSCHINSKEY, Memphis, TN (US)

(73) Assignee: DePuy Synthes Products, Inc., Raynham, MA (US)

(21) Appl. No.: 19/097,289

(22) Filed: Apr. 1, 2025

(51) Int. Cl. A61B 17/68; A61B 17/84; A61B 17/86; A61B 17/88; A61B 17/90

(52) U.S. Cl. CPC A61B 17/683; A61B 17/846; A61B 17/8625; A61B 17/8872; A61B 17/90; A61B 2017/681

(57) ABSTRACT

A system for providing bone movement can include a nail configured for placement across two bones. The nail can include a first end configured to be fixed in a first bone of the two bones, a second end configured to be fixed in a second bone of the two bones, and a first opening. The first opening can have a third end having a first diameter, a fourth end having a second diameter larger than the first diameter, and one or more walls connecting the third and fourth ends. The system can further include a first fixation device configured for delivery through the first opening and into the second bone, and configured to engage with the one or more walls of the first opening thereby moving the first and second bones in relation to one another.

### FIELD

The present disclosure generally relates to systems and methods for providing bone movement. More specifically, certain embodiments relate to systems and methods of moving a bone or bone fragment in relation to another bone or bone fragment.

### BACKGROUND

Placing intramedullary nails across bones or bone fragments can help ensure bones or bone fragments are disposed appropriately in relation to one another to aid in healing and recovery. A gap between bones or bone fragments, for example, as a result of a fracture or an osteotomy, can be adjusted using one or more types of compression, such as manual, dynamic, static, or external compression.

Accordingly, alternative systems and methods for providing bone movement, such as compression, across an osteotomy or fracture site would be useful.

### SUMMARY

The present disclosure is directed to systems and methods for providing bone movement.

An example system for providing bone movement is disclosed. The system can include a nail configured for placement across two bones. The nail can include a first end configured to be fixed in a first bone of the two bones, a second end configured to be fixed in a second bone of the two bones, and a first opening. The first opening can have a third end having a first diameter, a fourth end having a second diameter larger than the first diameter, and one or more walls connecting the third and fourth ends. The system can further include a first fixation device configured for delivery through the first opening and into the second bone, and configured to engage with the one or more walls of the first opening thereby moving the first and second bones in relation to one another.

Another example system for providing bone movement is disclosed. The system can include a nail configured for placement in a first bone. The nail can include a first end configured to be fixed in a first bone fragment of the first bone, a second end configured to be fixed in a second bone fragment of the first bone, and a first opening. The first opening can have a third end having a first diameter, and a fourth end having a second diameter larger than the first diameter. The system can further include a first fixation device configured for delivery through the first opening and into the second bone fragment by transitioning from the third end to the fourth end of the first opening thereby moving the first and second bone fragments in relation to one another.

An example method for moving a first bone in relation to a second bone is disclosed. The method can include delivering a nail into two bones such that a first end of the nail is fixed in a first bone and a second end of the nail is fixed in a second bone. The nail can include a first opening that itself can include a third end having a first diameter, and a fourth end having a second diameter larger than the first diameter. The method can include delivering a first fixation device through the first opening and into the second bone such that the first fixation device transitions from the third end of the first opening to the fourth end of the first opening thereby moving the first and second bones in relation to one another.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is an illustration of an example system for providing bone movement, according to aspects of the present disclosure.

FIG. 2 is a top view of an example component of a system for providing bone movement, according to aspects of the present disclosure.

FIGS. 3A-3B are side (3A) and top (3B) views of example components of a system for providing bone movement, according to aspects of the present disclosure.

FIGS. 4A-4B are side (4A) and top (4B) views of example components of a system for providing bone movement, according to aspects of the present disclosure.

FIG. 5 is an illustration of a step in an example method for providing bone movement, according to aspects of the present disclosure.

FIG. 6 is an illustration of a step in an example method for providing bone movement, according to aspects of the present disclosure.

FIG. 7 is an illustration of a step in an example method for providing bone movement, according to aspects of the present disclosure.

FIG. 8 is an illustration of a step in an example method for providing bone movement, according to aspects of the present disclosure.

FIG. 9 is an illustration of a step in an example method for providing bone movement, according to aspects of the present disclosure.

FIG. 10 is an illustration of a step in an example method for providing bone movement, according to aspects of the present disclosure.

FIG. 11 is an illustration of a step in an example method for providing bone movement, according to aspects of the present disclosure.

FIG. 12 is an illustration of a step in an example method for providing bone movement, according to aspects of the present disclosure.

FIG. 13A is a cross-sectional view of an example system for providing bone movement, according to aspects of the present disclosure.

FIG. 13B is a cross-sectional view of an example system for providing bone movement, according to aspects of the present disclosure.

FIG. 14 is an illustration of an example system for providing bone movement, according to aspects of the present disclosure.

FIG. 15 is an illustration of an example system for providing bone movement, according to aspects of the present disclosure.

FIG. 16 is a flowchart of an example method for providing bone movement, according to aspects of the present disclosure.

### DETAILED DESCRIPTION

The example systems and methods described herein generally involve providing bone movement. More specifically, certain embodiments relate to systems and methods of moving (e.g., compressing) a bone or bone fragment (e.g., in the hip, knee, foot, or hand) in relation to another bone or bone fragment to aid in patient healing. Providing such movement can allow for efficient adjustment of spacing between bones or bone fragments. Features from each example are combinable with other examples as understood by persons skilled in the pertinent art.

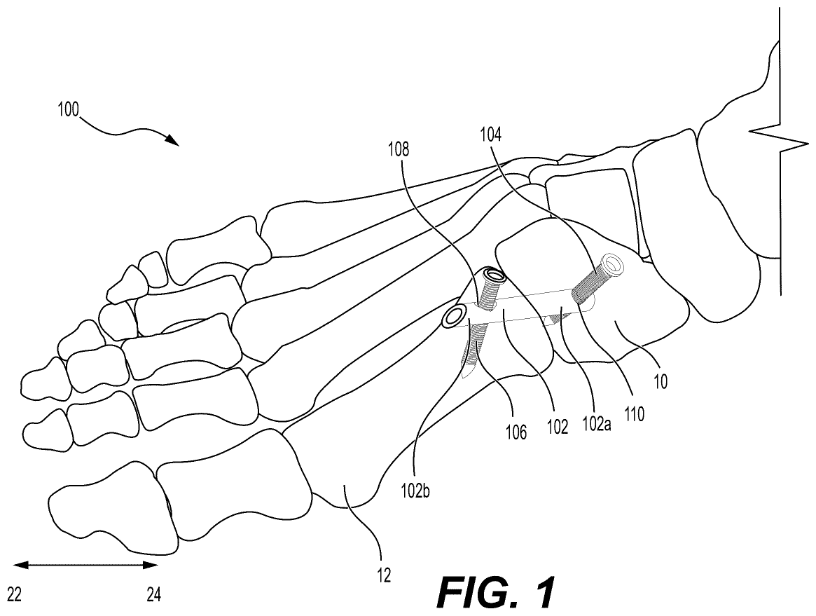

FIG. 1 is an illustration of an example system 100 for providing bone movement (e.g., compression) in an example environment. The environment can include a first bone 10 and a second bone 12, such as in a foot. The system 100 can include a nail 102 configured for placement across the first and second bones 10, 12. The nail can be placed across the first and second bones 10, 12 such that a first end 102a of the nail 102 is fixed in the first bone 10, while a second end 102b of the nail 102 is fixed in the second bone 12. In some embodiments, the nail 102 can be placed across two bone fragments as opposed to two separate bones, the bone fragments being a result of, for example, a fracture or an osteotomy. While FIG. 1, and other Figures throughout this disclosure, indicate the system is oriented along a first direction 22 (e.g., distal) and a second direction 24 (e.g., proximal), it should be understood that the nail 102 can be oriented in any direction within bones or bone fragments.

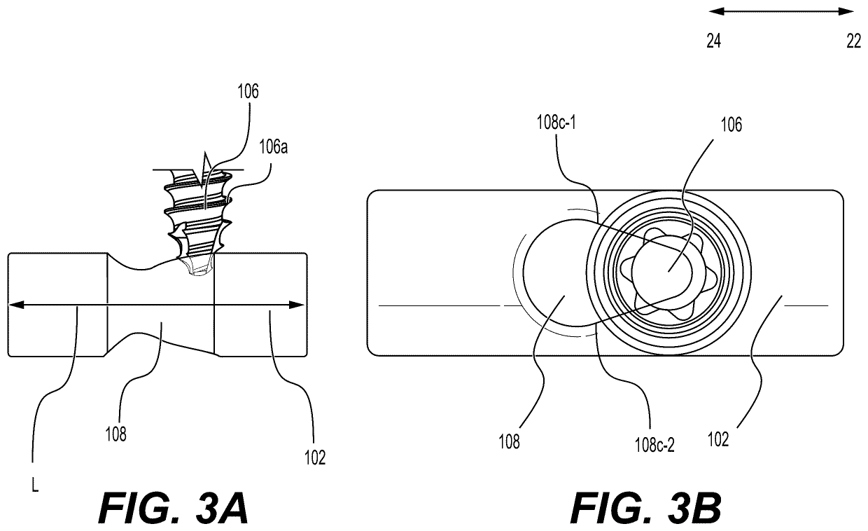

As further discussed below, the nail 102 can include a first opening 108 configured to engage with a first fixation device 106 of the system 100. The first fixation device 106 can be delivered through the first opening 108 and into the second bone 12, and can be configured to engage with one or more walls (e.g., walls 108c-1, 108c-2, FIG. 3A) of the first opening 108 to aid in moving the first and second bones 10, 12 in relation to one another.

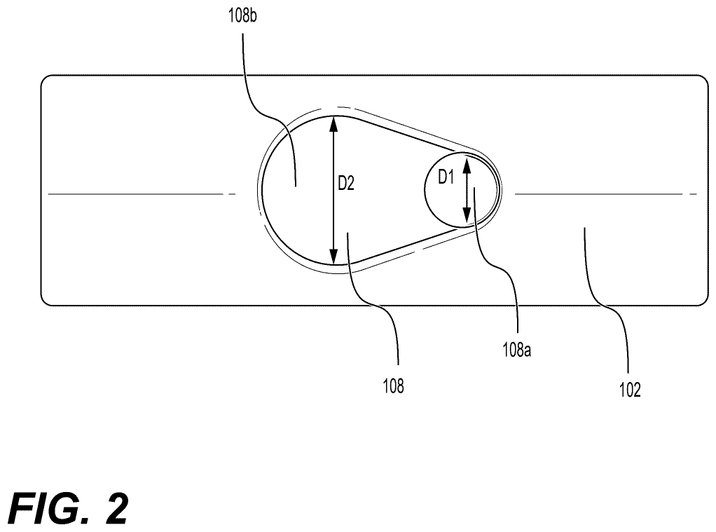

FIG. 2 provides a top view of the nail 102 and its first opening 108. The first opening 108 can include a first end 108a having a first diameter D1, and a second end 108b having a second diameter D2 that is larger than the first diameter D1. As shown, the first opening 108 can have a teardrop shape to aid in engagement with the first fixation device 106 and movement of the two bones relative to one another, as further discussed below. While the first opening 108 is depicted as having a teardrop shape, it should be understood that the first opening 108 can be configured to have a variety of shapes and/or orientations (e.g., oval, triangular, etc.).

FIGS. 3A-3B are side (3A) and top (3B) views of the nail 102 and first fixation device 106 of the system 100. As shown in FIG. 3A, the first opening 108 can run perpendicular to a longitudinal axis L of the nail 102. In some embodiments, however, first opening 108 can run non-perpendicular, or at an angle, to longitudinal axis L of the nail 102. When first delivering first fixation device 106 (e.g., a screw) into the first opening 108 of the nail 102, the first fixation device 106 can be driven through the first end 108a of the first opening 108. As the first fixation device 106 is driven or inserted (e.g., screwed) into the first end 108a of the first opening 108, one or more threads 106a of the first fixation device 106 can engage with one or more walls of the nail 102, such as wall 108c-1 and/or wall 108c-2. Nail 102 can have one or more walls 108c-1, 108c-2 that connect (e.g., tangentially) the first and second ends 108a, 108b of the first opening 108. While FIGS. 3A-3B, and other Figures throughout this disclosure, indicate the first end 108a of the first opening 108 being oriented in the second direction 24 while the second end 108b of the first opening 108 is oriented in the first direction 22, it should be understood that the first and second ends 108a, 108b of the first opening 108 can be oriented in any direction along the nail 102.

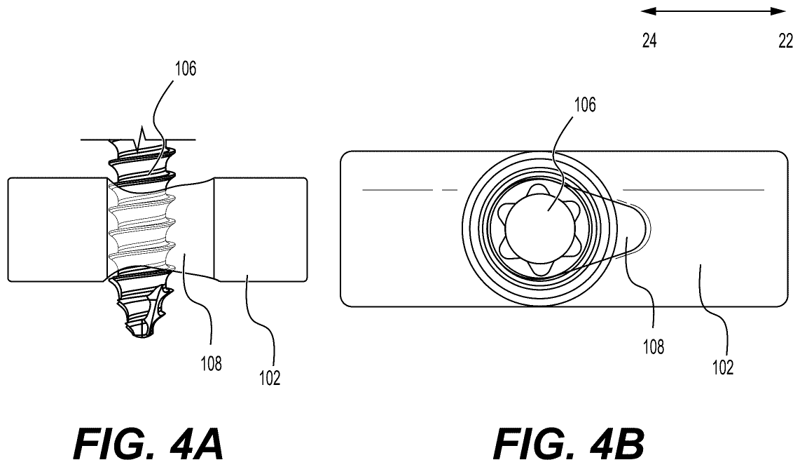

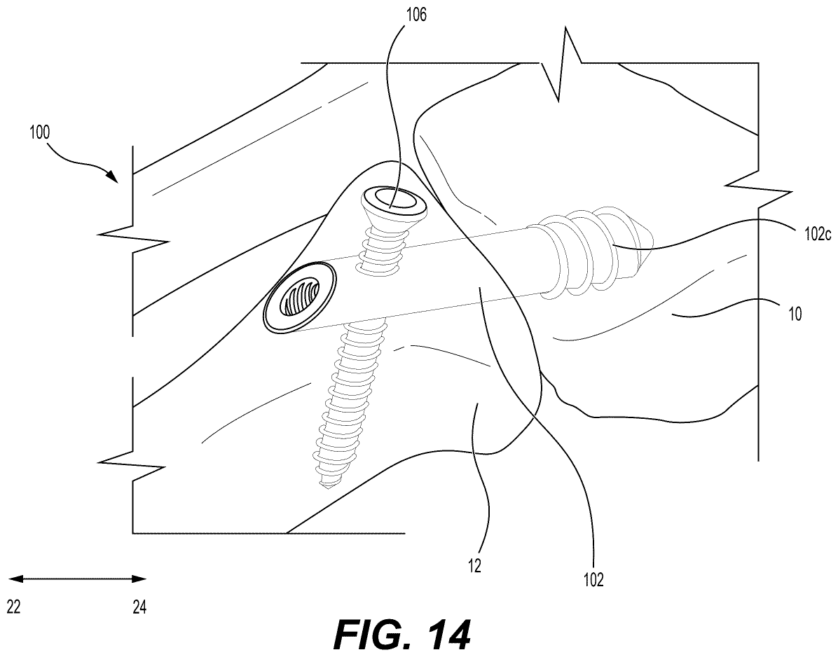

FIGS. 4A-4B are side (3A) and top (3B) views of the nail 102 and first fixation device 106 after the first fixation device 106 has been fully delivered (e.g., screwed) into the first opening 108. As the thread(s) 106a of the first fixation device 106 engage with one or more of the nail walls 108c-1, 108c-2 (FIG. 3B), this engagement pushes the first fixation device 106 away from the first end 108a of the first opening 108, and toward the second end 108b of the first opening 108. Once the first fixation device 106 has been fully delivered (e.g., screwed) into the first opening 108, the first fixation device 106 is aligned with the second end 108b of the first opening 108 (FIGS. 14A-14B).

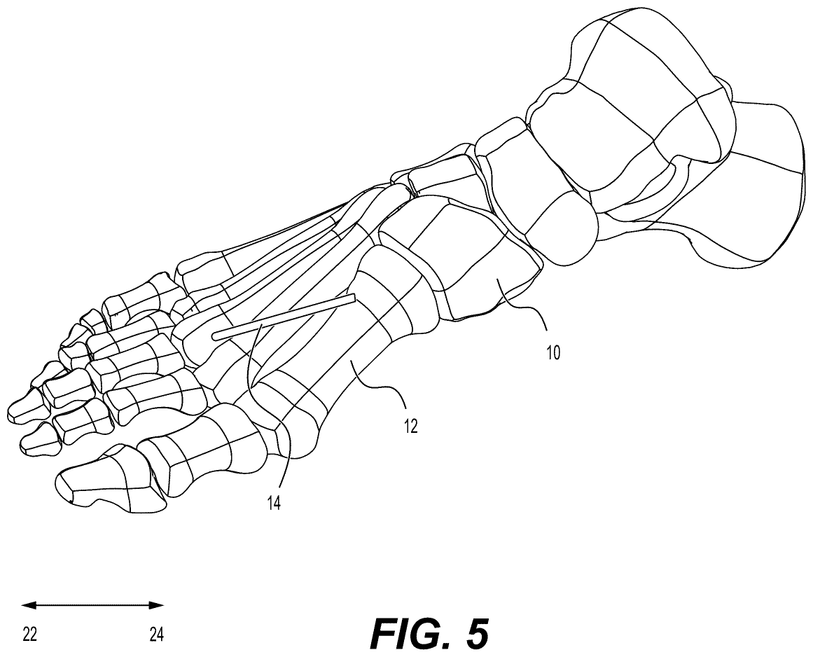

FIGS. 5-12 provide step-by-step illustrations of an example method for providing bone movement.

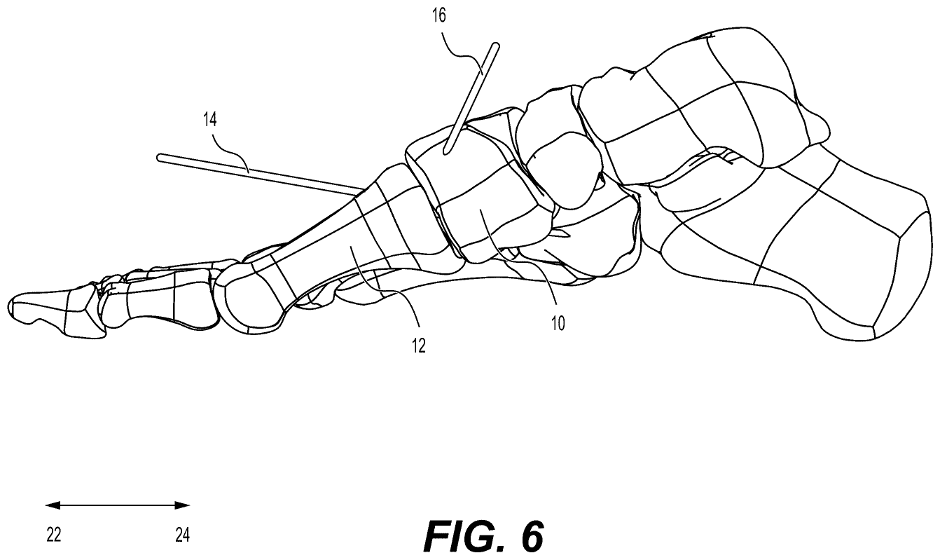

As shown in FIG. 5, the method can include delivering a first delivery device 14 (e.g., a Kirschner wire, also sometimes referred to as a K-wire) into the first and second bones 10, 12 by inserting the first delivery device 14 into the second bone 12 and then into the first bone 10. In some embodiments, after delivery of the first delivery device 14, the first delivery device 14 may extend outwardly from the first bone 10 and/or second bone 12, as shown in FIGS. 5 and 6. As further discussed below, the first delivery device 14 can aid in providing a delivery path for the nail 102 once the nail 102 is ready for delivery into the bone(s).

As shown in FIG. 6, the method can include delivering a second delivery device 16 (e.g., a Kirschner wire) into the first and second bones 10, 12 by inserting the second delivery device 16 into the first bone 10 and then into the second bone 12. In some embodiments, after delivery of the second delivery device 16, the second delivery device 16 may extend from the first bone 10 and/or second bone 12. The second delivery device 16 can aid in stabilizing the first and second bones 10, 12 relative to one another and/or provide a way for a user (e.g., a physician) to hold onto the bones or bone fragments while conducting the disclosed methods.

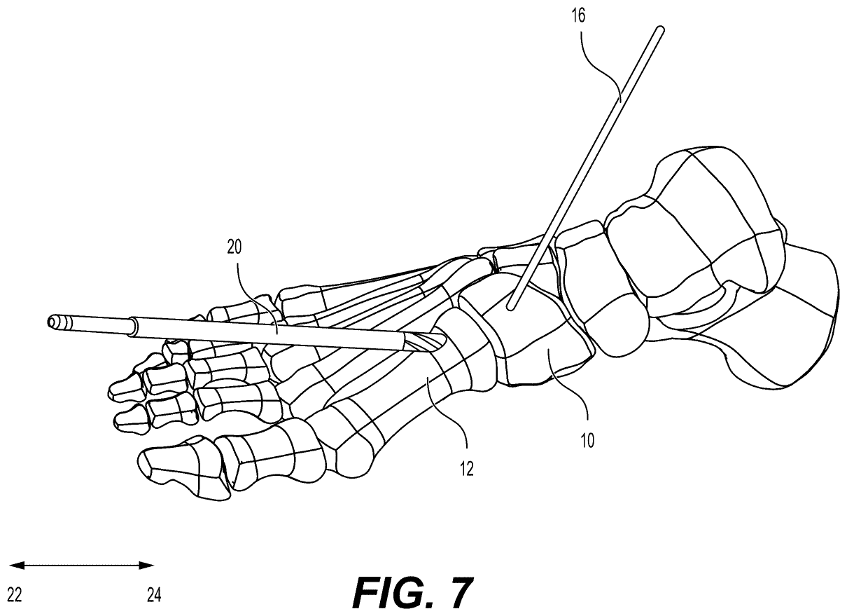

As shown in FIG. 7, the method can include engaging a third delivery device 20 (e.g., a reamer) with the first delivery device 14 thereby generating a delivery path for the nail 102 into the two bones 10, 12. For example, after the first delivery device 14 has provided a targeted delivery path for the nail, the reamer can be used to drill a larger delivery path using the first delivery device 14 as a guide.

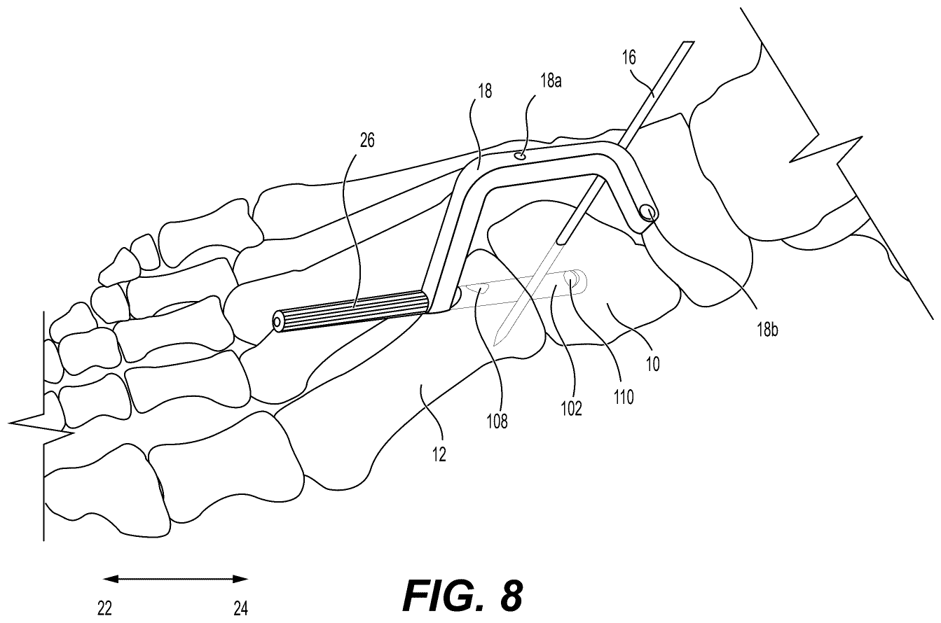

As shown in FIG. 8, the method can include delivering the nail 102 into the delivery path (FIG. 7) such that a first end 102a of the nail 102 is fixed in the first bone 10, and a second end 102b of the nail 102 is fixed in the second bone 12 (FIG. 1). The method can then include engaging a guide arm 18 with the nail 102 (e.g., by locking the guide arm 18 to the nail 102 via a locking knob 26) such that a first opening 18a of the guide arm 18 is aligned with the first end 108a of the first opening 108 of the nail 102. This alignment can provide a pilot hole to aid in ensuring that when delivering the first fixation device 106 into the first opening 108, the first fixation device 106 is properly aligned with the center of the first end 108a of the first opening 108, as discussed above with respect to FIGS. 2 and 3A-3B.

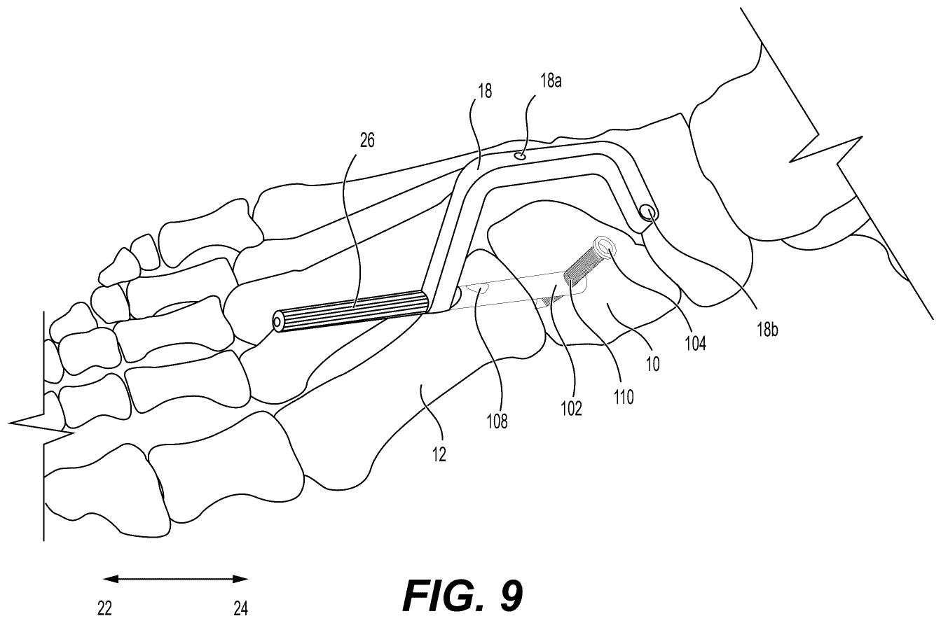

As shown in FIG. 9, in some embodiments, the nail 102 can include a second opening 110 configured to engage with a second fixation device 104 (also shown in FIG. 1). The second fixation device 104 can be delivered (e.g., screwed) into and through the second opening 110 and into the first bone 10 to provide additional stability of the nail 102 in the first bone 10. As shown in FIG. 9, the guide arm can include a second opening 18b aligned with the second opening 110 of the nail 102 to aid in properly aligning the second fixation device 104 with the second opening 110 of the nail and/or drilling a pilot hole into the second opening 110 to provide a delivery path for the second fixation device 104 into the second opening 110. FIG. 9 shows the step of delivering the second fixation device 104 into the second opening 110 to provide stability to the nail 102 in the first bone 10. The second delivery device 16 can also be removed at this step.

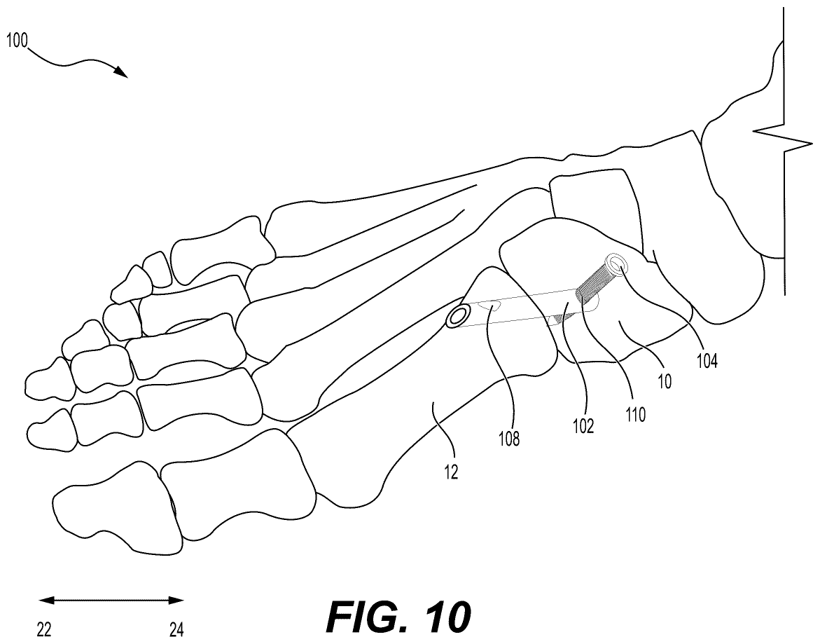

As shown in FIG. 10, the method can include removing the guide arm 18 and locking knob 26, leaving behind the nail 102 and second fixation device 104 in the bones.

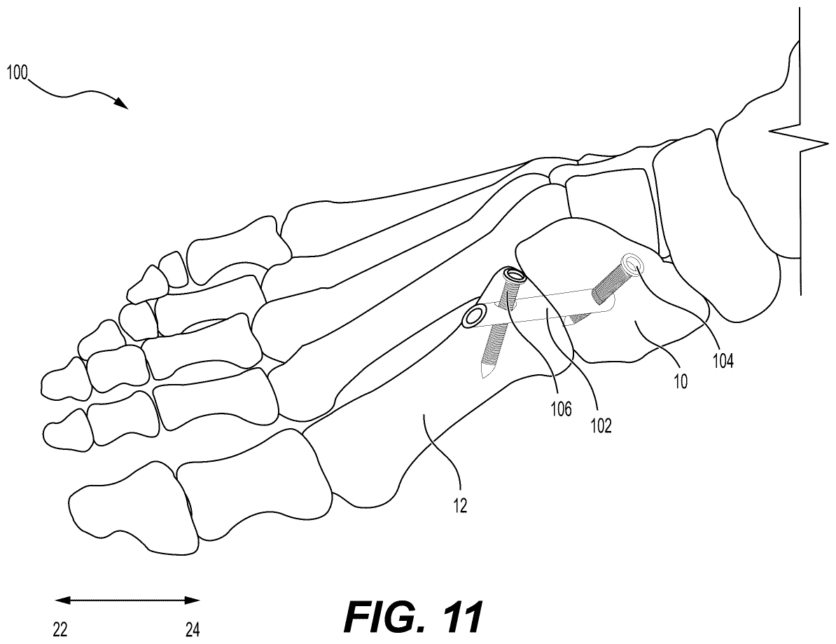

As shown in FIG. 11, the method can include delivering the first fixation device 106 into the first opening 108 of the nail 102. As discussed herein, as the first fixation device 106 is delivered (e.g., screwed) into the first opening 108 at the first end 108a, the first fixation device 106 transitions from the first end 108a of the opening 108 to the second end 108b of the opening 108 thereby moving the first and second bones 10, 12 relative to one another.

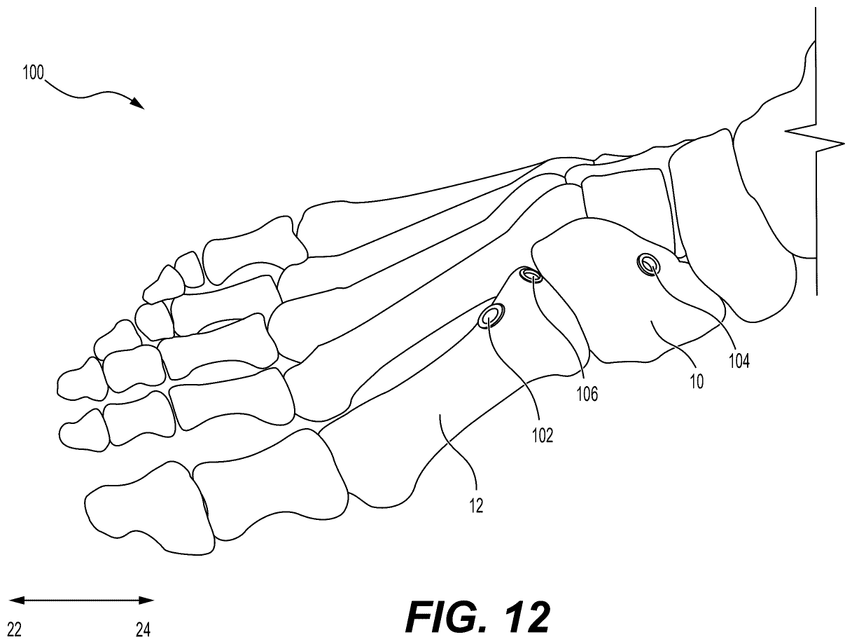

As shown in FIG. 12, once the first fixation device 106 is fixed in the nail 102, at least a portion of the nail 102, first fixation device 106, and/or second fixation device 104 can be left at least partially exposed or extended outwardly from the first and second bones 10, 11.

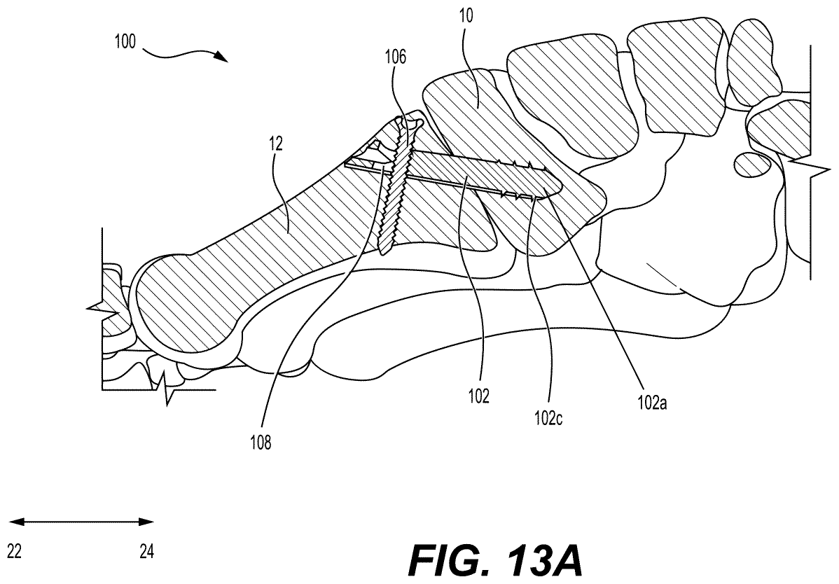

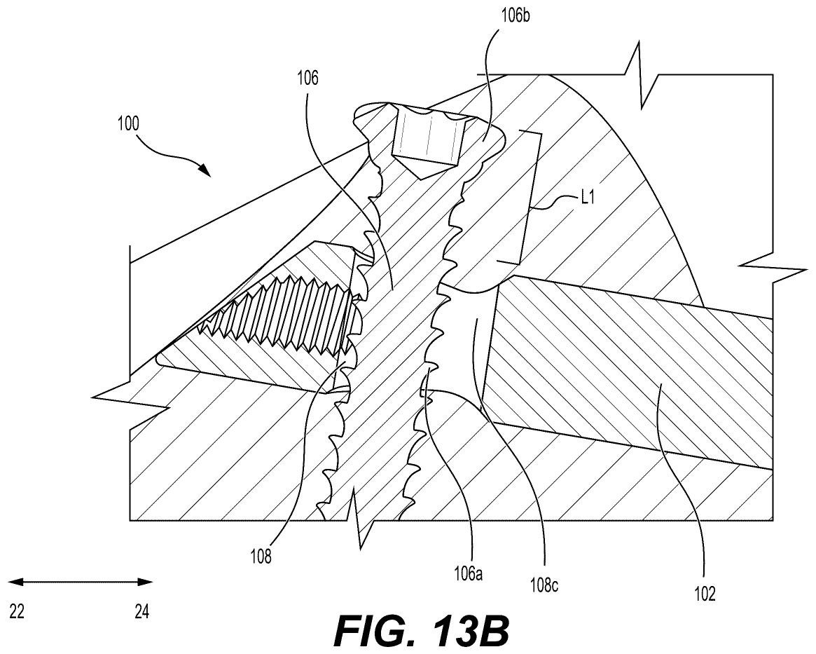

FIG. 13A is a cross-sectional view of an alternative example system 100 for providing bone movement. As shown, many of the features are the same as or similar to those discussed throughout this disclosure. In some embodiments, however, the nail 102 can include one or more threads 102c to aid in fixating the first end 102a of the nail 102 in the first bone 10. A nail 102 including such threads 102c may not also have a second opening 110 for engaging with a second fixation device 104.

FIG. 13B is a cross-sectional view of the system shown in FIG. 13A. As shown, in some embodiments, once the first fixation device 106, (e.g., a screw), is fully engaged with (e.g., screwed into) the nail 102, a head 106b of the screw 106 may be situated at a distance L1 from the nail 102. In other embodiments, however, the screw head 106b may engage with a portion of the nail 102. It should be understood that this feature can also be included in a system including a nail 102 having a second opening 110 for engaging a second fixation device 104 (e.g., FIG. 1).

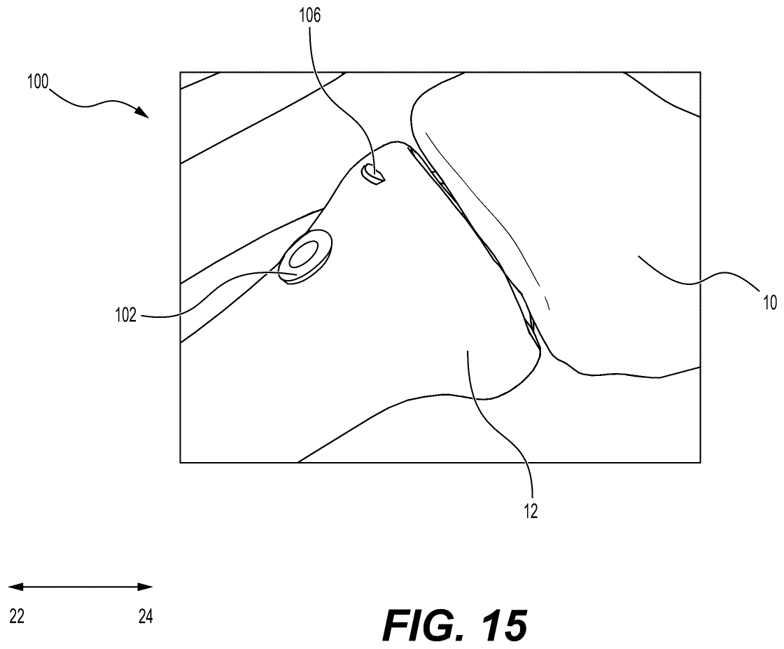

FIGS. 14 and 15 provide perspective views of the alternative system shown in FIG. 13A. FIG. 14 illustrates an example of how the nail 102 can engage with the first and second bones 10, 12 with the first fixation device 106 delivered through the first opening 108 of the nail 102. FIG. 15 illustrates how at least a portion of the nail 102 and/or first fixation device 106 can extend outwardly (or be exposed) from the second bone 12 once fixed in place.

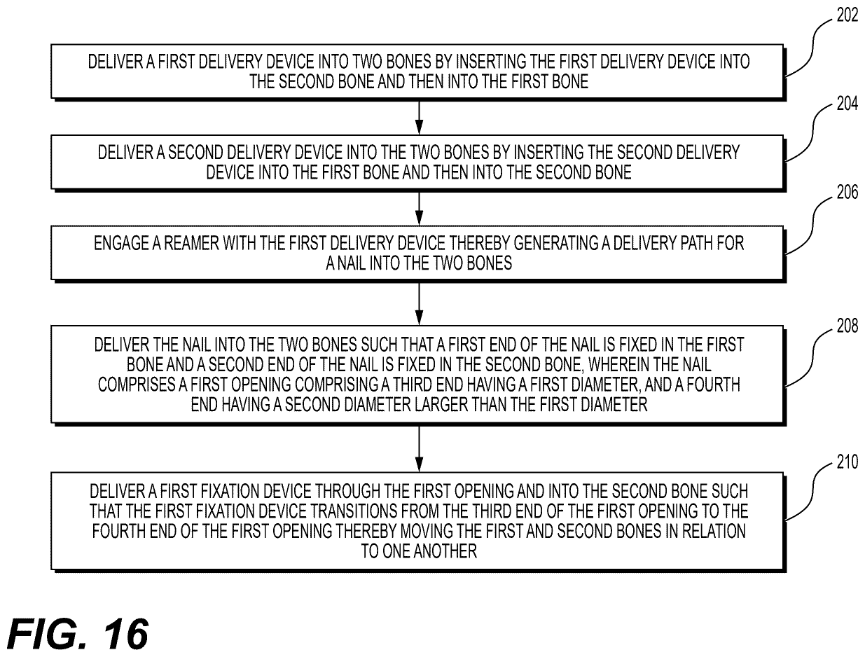

FIG. 16 is a flowchart of an example method for providing bone movement.

In block 202, the method can include delivering a first delivery device (e.g., first delivery device 14) into two bones (e.g., first bone 10 and second bone 12) by inserting the first delivery device into a second bone (e.g., second bone 12) and then into a first bone (e.g., first bone 10). This step may be the same as or similar to the step described above with respect to FIG. 5.

In block 204, the method can include delivering a second delivery device (e.g., second delivery device 16) into the two bones by inserting the second delivery device into the first bone and then into the second bone. This step may be the same as or similar to the step described above with respect to FIG. 6.

In block 206, the method can include engaging a reamer (e.g., reamer 20) with the first delivery device thereby generating a delivery path for delivering a nail (e.g., nail 102) into the two bones. This step may be the same as or similar to the step described above with respect to FIG. 7.

In block 208, the method can include delivering the nail into the two bones such that a first end (e.g., first end 102a) of the nail is fixed in the first bone and a second end (e.g., second end 102b) of the nail is fixed in the second bone. This step may be the same as or similar to the step described above with respect to FIG. 8.

In block 210, the method can include delivering a first fixation device (e.g., first fixation device 106) through a first opening (e.g., first opening 108) of the nail and into the second bone such that the first fixation device transitions from a first end (e.g., first end 108a) of the first opening to a second end (e.g., second end 108b) of the first opening thereby moving the first and second bones in relation to one another, as discussed herein.

The descriptions contained herein are examples of embodiments of the disclosure and are not intended in any way to limit the scope of the disclosure. As described herein, the disclosure contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

The disclosed technology described herein can be further understood according to the following clauses.

Clause 1: A system for providing bone movement, the system comprising: a nail configured for placement across two bones and comprising: a first end configured to be fixed in a first bone of the two bones; a second end configured to be fixed in a second bone of the two bones; and a first opening comprising: a third end having a first diameter; a fourth end having a second diameter larger than the first diameter; and one or more walls connecting the third and fourth ends; and a first fixation device configured for delivery through the first opening and into the second bone, and configured to engage with the one or more walls of the first opening thereby moving the first and second bones in relation to one another.

Clause 2: The system of clause 1, wherein the nail further comprises a second opening, and wherein the system further comprises a second fixation device configured for delivery through the second opening and into the first bone.

Clause 3: The system of clause 1, wherein the first opening has a teardrop shape.

Clause 4: The system of clause 1, wherein the first fixation device transitions from the third end of the first opening to the fourth end of the first opening as the first fixation device is delivered through the first opening.

Clause 5: The system of clause 1, wherein the first fixation device engages with the one or more walls of the first opening via one or more threads of the first fixation device.

Clause 6: The system of clause 1, wherein when the first and second bones are moved in relation to one another, the first fixation device engages with the third end of the first opening.

Clause 7: The system of clause 1, wherein when the first and second bones are moved in relation to one another, at least a respective portion of the nail and the first fixation device are disposed external to the first and second bones.

Clause 8: The system of clause 1, wherein the first fixation device comprises a screw.

Clause 9: The system of clause 8, wherein the screw comprises a head, and wherein when the first and second bones are moved in relation to one another, the head of the screw is disposed at least a first distance from the nail.

Clause 10: The system of clause 1, wherein the nail comprises one or more threads configured to engage with the first bone.

Clause 11: The system of clause 1, wherein the first opening is perpendicular to a longitudinal axis of the nail.

Clause 12: The system of clause 1, wherein the first opening is disposed at an angle in relation to a longitudinal axis of the nail.

Clause 13: A system for providing bone movement, the system comprising: a nail configured for placement in a first bone and comprising: a first end configured to be fixed in a first bone fragment of the first bone; a second end configured to be fixed in a second bone fragment of the first bone; and a first opening comprising: a third end having a first diameter; and a fourth end having a second diameter larger than the first diameter; and a first fixation device configured for delivery through the first opening and into the second bone fragment by transitioning from the third end to the fourth end of the first opening thereby moving the first and second bone fragments in relation to one another.

Clause 14: The system of clause 13, wherein the first opening has a teardrop shape.

Clause 15: The system of clause 13, wherein the first fixation device comprises a screw, wherein the screw comprises a head, and wherein when the first and second bone fragments are moved in relation to one another, the head of the screw is disposed at least a first distance from the nail.

Clause 16: A method for moving a first bone in relation to a second bone, the method comprising: delivering a nail into two bones such that a first end of the nail is fixed in a first bone and a second end of the nail is fixed in a second bone, wherein the nail comprises a first opening comprising: a third end having a first diameter; and a fourth end having a second diameter larger than the first diameter; and delivering a first fixation device through the first opening and into the second bone such that the first fixation device transitions from the third end of the first opening to the fourth end of the first opening thereby moving the first and second bones in relation to one another.

Clause 17: The method of clause 16, further comprising: engaging a guide arm with the nail such that a second opening of the guide arm is aligned with the third end of the first opening of the nail, wherein delivering the first fixation device through the first opening comprises utilizing the second opening of the guide arm to provide a pilot hole into the third end of the first opening.

Clause 18: The method of clause 17, further comprising: delivering a first delivery device into the two bones by inserting the first delivery device into the second bone and then into the first bone; delivering a second delivery device into the two bones by inserting the second delivery device into the first bone and then into the second bone; and engaging a reamer with the first delivery device thereby generating a delivery path for the nail into the two bones, wherein the first and second delivery devices comprise Kirschner wires.

Clause 19: The method of clause 16, wherein the nail further comprises a second opening, the method further comprising: delivering a second fixation device through the second opening and into the first bone.

Clause 20: The method of clause 16, wherein the first opening further comprises one or more walls, wherein the first fixation device comprises one or more threads, and wherein delivering the first fixation device through the first opening comprises engaging the one or more threads of the first fixation device with the one or more walls of the first opening.

What is claimed is:

1. A system for providing bone movement, the system comprising:
a nail configured for placement across two bones and comprising:
a first end configured to be fixed in a first bone of the two bones;
a second end configured to be fixed in a second bone of the two bones; and
a first opening comprising:
a third end having a first diameter;
a fourth end having a second diameter larger than the first diameter; and
one or more walls connecting the third and fourth ends; and
a first fixation device configured for delivery through the first opening and into the second bone, and configured to engage with the one or more walls of the first opening thereby moving the first and second bones in relation to one another.

2. The system of claim 1, wherein the nail further comprises a second opening, and wherein the system further comprises a second fixation device configured for delivery through the second opening and into the first bone.

3. The system of claim 1, wherein the first opening has a teardrop shape.

4. The system of claim 1, wherein the first fixation device transitions from the third end of the first opening to the fourth end of the first opening as the first fixation device is delivered through the first opening.

5. The system of claim 1, wherein the first fixation device engages with the one or more walls of the first opening via one or more threads of the first fixation device.

6. The system of claim 1, wherein when the first and second bones are moved in relation to one another, the first fixation device engages with the third end of the first opening.

7. The system of claim 1, wherein when the first and second bones are moved in relation to one another, at least a respective portion of the nail and the first fixation device are disposed external to the first and second bones.

8. The system of claim 1, wherein the first fixation device comprises a screw.

9. The system of claim 8, wherein the screw comprises a head, and wherein when the first and second bones are moved in relation to one another, the head of the screw is disposed at least a first distance from the nail.

10. The system of claim 1, wherein the nail comprises one or more threads configured to engage with the first bone.

11. The system of claim 1, wherein the first opening is perpendicular to a longitudinal axis of the nail.

12. The system of claim 1, wherein the first opening is disposed at an angle in relation to a longitudinal axis of the nail.

13. A system for providing bone movement, the system comprising:
a nail configured for placement in a first bone and comprising:
a first end configured to be fixed in a first bone fragment of the first bone;
a second end configured to be fixed in a second bone fragment of the first bone; and
a first opening comprising:
a third end having a first diameter; and
a fourth end having a second diameter larger than the first diameter; and
a first fixation device configured for delivery through the first opening and into the second bone fragment by transitioning from the third end to the fourth end of the first opening thereby moving the first and second bone fragments in relation to one another.

14. The system of claim 13, wherein the first opening has a teardrop shape.

15. The system of claim 13, wherein the first fixation device comprises a screw, wherein the screw comprises a head, and wherein when the first and second bone fragments are moved in relation to one another, the head of the screw is disposed at least a first distance from the nail.

16. A method for moving a first bone in relation to a second bone, the method comprising:
delivering a nail into two bones such that a first end of the nail is fixed in a first bone and a second end of the nail is fixed in a second bone,
wherein the nail comprises a first opening comprising:
a third end having a first diameter; and
a fourth end having a second diameter larger than the first diameter; and
delivering a first fixation device through the first opening and into the second bone such that the first fixation device transitions from the third end of the first opening to the fourth end of the first opening thereby moving the first and second bones in relation to one another.

17. The method of claim 16, further comprising:
engaging a guide arm with the nail such that a second opening of the guide arm is aligned with the third end of the first opening of the nail,
wherein delivering the first fixation device through the first opening comprises utilizing the second opening of the guide arm to provide a pilot hole into the third end of the first opening.

18. The method of claim 17, further comprising:
delivering a first delivery device into the two bones by inserting the first delivery device into the second bone and then into the first bone;
delivering a second delivery device into the two bones by inserting the second delivery device into the first bone and then into the second bone; and
engaging a reamer with the first delivery device thereby generating a delivery path for the nail into the two bones,
wherein the first and second delivery devices comprise Kirschner wires.

19. The method of claim 16, wherein the nail further comprises a second opening, the method further comprising:
delivering a second fixation device through the second opening and into the first bone.

20. The method of claim 16, wherein the first opening further comprises one or more walls, wherein the first fixation device comprises one or more threads, and wherein delivering the first fixation device through the first opening comprises engaging the one or more threads of the first fixation device with the one or more walls of the first opening.
